(19) United States
(12) Patent Application Publication
(10) Pub. No.: US 2008/0317313 A1
(43) Pub. Date: Dec. 25, 2008

(54) SYSTEM AND METHOD FOR TRACKING MOTION FOR GENERATING MOTION CORRECTED TOMOGRAPHIC IMAGES

(75) Inventors: James S. Goddard, Knoxville, TN (US); Justin S. Baba, Knoxville, TN (US)

Correspondence Address: AKERMAN SENTERFITT, P.O. BOX 3188, WEST PALM BEACH, FL, 33402-3188, US

(73) Assignee: UT-Battelle, LLC, Oak Ridge, TN (US)

(21) Appl. No.: 12/117,213

(22) Filed: May 8, 2008

Related U.S. Application Data
(63) Continuation-in-part of application No. 11/241,359, filed on Sep. 30, 2005.

(51) Int. Cl. A61B 5/055

(52) U.S. Cl. 382/131; 600/410

(57) ABSTRACT

A method and related system for generating motion corrected tomographic images includes the steps of illuminating a region of interest (ROI) to be imaged being part of an unrestrained live subject and having at least three spaced apart optical markers thereon. At least one camera is used to obtain images of the markers. Motion data comprising 3D position and orientation of the markers relative to an initial reference position is then calculated. The at least three spaced apart optical markers and the at least one camera are sufficient in quantity and position to avoid multiple epipolar solutions. Motion corrected tomographic data obtained from the ROI using the motion data is then obtained, where motion corrected tomographic images obtained therefrom.

### CROSS-REFERENCE TO RELATED APPLICATIONS

This application is a continuation-in-part of U.S. application Ser. No. 11/241,359, entitled “System and Method for Generating Motion Corrected Tomographic Images,” filed Sep. 30, 2005, the entirety of which is incorporated herein by reference.

### STATEMENT REGARDING FEDERALLY SPONSORED RESEARCH OR DEVELOPMENT

The United States Government has rights in this invention pursuant to Contract No. DE-AC05-00OR22725 between the United States Department of Energy and UT-Battelle, LLC.

### BACKGROUND OF THE INVENTION

Patient motion or motion of a living subject during imaging can cause image artifacts. The sources of motion include restlessness, as well as, processes such as respiration and heart beats, which can produce small movements due to pressure changes over the cardiac cycle. In some cases motion artifacts degrade the diagnostic value of an image.

Efficient methods for testing new drugs are very important to the pharmaceutical industry. The ability to screen test subjects for effects of a particular drug is an essential element in the process of product development. Small animals are essential for pharmaceutical testing, and mice in particular are useful for modeling human diseases. Efforts to scale down clinical medical imaging systems for smaller subjects have allowed medical researchers to obtain high-resolution computed tomography (CT) images of small animals for disease studies. Noninvasive imaging techniques, such as X-ray CT and positron emission tomography (PET), have been developed for small animal medical imaging applications. For example, small animal imaging is used in cancer research to monitor tumor growth and regression in mice.

While anatomical models are useful for studying drug effectiveness, it is very often desirable to screen test subjects for physiological effects of a drug. PET and single photon emission computed tomography (SPECT) are among current techniques used for functional medical imaging. Because test subjects must be kept alive during the screening process in order to monitor functional processes, either the animal must remain motionless for the duration of the scan or its movements must be measured and recorded with a high degree of precision and accuracy. Although sedation and physical restraint can be used to impede animal motion for this type of medical scan, both methods have the potential to alter the neurological and physiological processes that are being studied. Unrestrained awake animals tend to sometimes move rapidly. Unfortunately, existing measurement systems are not designed for fast motion measurement and correction.

### SUMMARY OF THE INVENTION

A method and related system for generating motion corrected tomographic images. The method includes illuminating a region to of interest (ROI) to be imaged being part of a live subject and having at least three spaced apart optical markers thereon. Optical images of the markers can be obtained from at least one camera. Motion data comprising 3D position and orientation of the markers relative to an initial reference position can be calculated. The at least three spaced apart optical markers and the at least one camera can be sufficient in quantity and position to avoid multiple epipolar solutions. Motion correcting tomographic data obtained from the ROI using said motion data can be obtained and used to produce motion corrected tomographic images.

In some embodiments, the method can include illuminating a region of interest (ROI) to be tomographically imaged. The ROI can be part of an unrestrained subject having at least three spaced apart retro-reflective optical markers attached thereto. The markers are proximate the ROI and each marker is either polarizing or depolarizing for an illuminating wavelength. Filtered optical images of the markers are acquired from at least one filtered camera. A polarization filter on the filtered camera(s) enables selective detection of illumination reflected by the at least three optical markers. Motion data, including 3D position and orientation of the markers relative to an initial reference position, is calculated. The at least three spaced apart retro-reflective optical markers and the at least one filtered camera are sufficient in quantity and position to avoid multiple epipolar solutions. Finally, the motion data is used to motion correct tomographic data of the ROI obtained simultaneously with the motion data and the corrected tomographic data is used to produce motion corrected tomographic images. At least one of the filtered cameras can be a video camera.

The animal can be disposed in a confinement volume which is optically transparent to the illumination wavelength used in method. The motion corrected tomographic images can be single photon emission computed tomography (SPECT) images. The illumination can be aligned to be approximately coaxial with an optical axis of said first camera.

The illuminating can be strobed illuminating. The acquisition of the optical images can be synchronized to a strobed pulse of illumination to cause acquisition of the optical images during a pulsed illumination period. The calculating motion data step can include processing the images using a combination of segmentation, object feature extraction and digital filtering.

The method can include at least a fourth spaced apart retro-reflective optical marker. The at least four retro-reflective markers can be arranged to eliminate multiple epipolar solutions.

The method can include first and second filtered cameras and the acquiring step can include acquiring simultaneous images from the first and second filtered cameras. The calculating motion data step can include processing the simultaneous images using a combination of segmentation, object feature extraction and digital filtering. The method can include first, second, and third filtered cameras and the acquiring step can include acquiring simultaneous images of at least three of the spaced apart retro-reflective optical markers from at least two of the first, second, and third filtered cameras.

The illumination can be polarized. At least two of the retro-reflective markers can be polarized and have different polarization characteristics.

In one system embodiment, the system for obtaining a motion correcting tomography-based imaging system includes at least three spaced apart optical markers for placement on a region of interest (ROI) to be imaged, at least one radiation detector, a first processor communicably connected to the radiation detector, a structure for positioning the radiation detector relative to the ROI, and a motion correcting system. The radiation detector can be capable of collecting (i) radiation data emitted from a radioactive isotope in the ROI or (ii) radiation data provided by the ROI attenuating radiation provided by an external radiation source. The motion correcting system can include at least one illumination source for illuminating the ROI, at least one camera for acquiring images of the markers, and at least a second processor communicably connected to said first processor for calculating motion data comprising 3D position and orientation of the markers relative to an initial reference position and motion correcting the radiation data. The at least second processor is capable of producing motion corrected tomographic images from the motion correcting radiation data. The system can include at least two cameras, at least four optical markers, or both, in order to avoid multiple epipolar solutions.

In some embodiments, the invention is drawn to a motion correcting tomography-based imaging system. The system includes at least three spaced apart retro-reflective optical markers for placement on a ROI to be imaged, at least one radiation detector, a structure for positioning the at least one radiation detector and a motion correcting system. Each of the markers is either polarizing or depolarizing for a wavelength produced by an illumination source. The radiation detector can be for collecting (i) radiation data emitted from a radioactive isotope in the ROI or (ii) radiation data provided by the ROI attenuating radiation provided by an external radiation source. The a first processor can be communicably connected to said radiation detector.

The motion correcting system can include at least one illumination source for illuminating the ROI; at least one filtered camera for acquiring images from the markers; a structure for positioning the at least one filtered camera; and at least a second processor. The at least second processor can be communicably connected to the first processor for calculating motion data including 3D position and orientation of the retro-reflective markers relative to an initial reference position, and motion correcting the radiation data. Motion corrected tomographic images can be obtained from the motion correcting radiation data. The system includes at least two filtered cameras, at least four spaced apart retro-reflective optical markers, or both, in order to avoid multiple epipolar solutions.

The system can be a single photon emission computed tomography (SPECT) system. The illuminating can be aligned to be approximately coaxial with at least one of said at least one filtered cameras. The at least one illumination source can provide strobed illumination. Acquisition of the images can be synchronized to a strobe pulse to cause the simultaneous acquisition during an illumination period. The at least one radiation detector comprises a first and a second detector.

### BRIEF DESCRIPTION OF THE DRAWINGS

A fuller understanding of the present invention and the features and benefits thereof will be obtained upon review of the following detailed description together with the accompanying drawings, in which:

FIG. 1 is an image of a mouse in a burrow, where the mouse has three optical markers applied to its head.

FIG. 2 is a digitized image showing regions identified as optical markers and specular reflection.

FIG. 3 is a schematic diagram of an exemplary motion correcting single photon emission computed tomography (SPECT) imaging system, having two cameras and two radiation detectors.

FIG. 4 is a communication flow diagram for system components for the system shown in FIG. 3.

FIG. 5 shows a digitized image of the mouse in FIG. 1 with the retro reflectors from each camera and with tracking enabled. The markers are outlined and numbered showing that they have been segmented and that correspondence has been determined. In this image, the lines between the markers indicates that successful model fitting has been achieved and that a full 3D transformation has been calculated between the camera reference frame and the model reference frame.

FIG. 6 shows impinging illumination being reflected to a camera by both the confinement volume and an optical marker.

FIG. 7 shows the orientation of illumination as it passes through a series of filters.

FIG. 8 shows how specular reflections can be eliminated using polarizing illumination and retro-reflective optical markers with a polarized or polarization rotated polarization characteristic.

### DETAILED DESCRIPTION

As noted above, high quality 3D images from conventional scanned data requires that the object or other structure to be imaged remain stationary during the scan. However, imaging unrestrained live subjects, such as animals (e.g. rats), presents difficulties during scans and significantly reduces the quality of the resulting 3D images. The method disclosed herein corrects for motion during the scan, thus improving the quality of 3D images obtained.

The method can include illuminating a region of interest (ROI) to be tomographically imaged, wherein the ROI is part of an unrestrained subject 160 having at least three spaced apart retro-reflective optical markers 171 attached thereto. The optical markers 171 are proximate the ROI and each marker 171 can be either polarizing or depolarizing for an illuminating wavelength. Filtered optical images of the markers 171 are acquired from at least one filtered camera 116. A polarization filter 240 on a camera(s) 116 can enable selective detection of illumination reflected by the at least three optical markers 171. Motion data, including 3D position and orientation of the markers 171 relative to an initial reference position, is calculated. The at least three spaced apart retro-reflective optical markers 171 and the at least one filtered camera 116 are sufficient in quantity and position to avoid multiple epipolar solutions. Finally, the motion data is used to motion correct tomographic data of the ROI obtained simultaneously with the motion data and the corrected tomographic data is used to produce motion corrected tomographic images. At least one filtered cameras can be a video camera.

The at least three spaced apart retro-reflective optical markers and the at least one filtered camera are sufficient in quantity and position to avoid multiple epipolar solutions. This includes any quantity and positioning that allows for the three dimensional location of each of the markers to be resolved without ambiguity. Generally, this will require that the method and system described herein include at least two cameras 116, at least four optical markers 171, or both. The method of calculating an epipolar solution is explained in more detail below.

Where at least four optical markers are used, it is possible to avoid multiple epipolar solutions using a single camera if the at least four optical markers are attached to the ROI in a spatially unambiguous pattern where the inter-marker positioning is known. A spatially unambiguous pattern is one that cannot be oriented to produce the same pattern in multiple orientations. For example, a triangle with markers at each of the three corners and a fourth marker along an edge of the triangle would be spatially unambiguous, whereas a square or an equilateral triangle with markers at each of the corners would not generally be a spatially unambiguous pattern. Asymmetric arrangements would generally be spatially unambiguous. Similarly, a spatially unambiguous pattern may be achieved in any shape by using markers with different polarization characteristics. The inter-marker positioning can be determined using any known technique, including using epipolar techniques to analyze simultaneous images from two or more cameras.

The subject 160 can be disposed in a confinement volume 112 which is optically transparent to the illumination wavelength. The motion corrected tomographic images can be single photon emission computed tomography (SPECT) images. The illumination can be aligned to be approximately coaxial with an optical axis of said first camera.

As used herein, “approximately coaxially,” is used to indicate that the path of the illumination travels in the direction of the optical axis of the first camera at an angle that deviates about 20 degrees or less from parallel to the optical axis, or deviates about 10 degrees or less from parallel, or deviates about 5 degrees or less from parallel. For example, illumination can be approximately coaxial where it originates from LEDs arranged in a ring around a camera lens where the LEDs are focused on the subject 160, the confinement volume 112, or both.

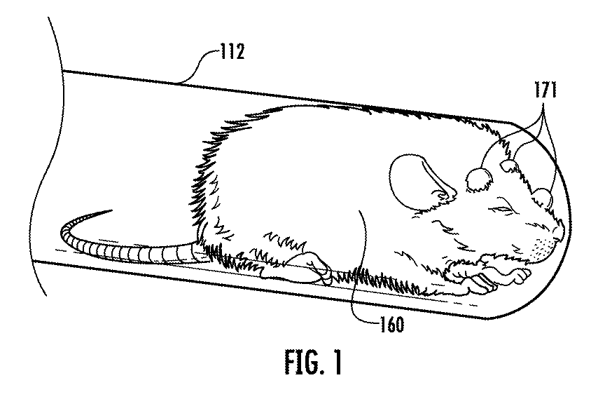

As used herein, “selective detection” is used to indicate detection that is able to distinguish between detected illumination that was reflected by a marker 171 and illumination reflected by, or originating, from other sources. As shown in FIG. 1, the subject can be a mouse that has three optical markers 171 glued to its head and is confined to an optically transparent test tube. In such an arrangement, “selective detection” allows the cameras to distinguish illumination reflected by the markers from specular reflections of the optically transparent confinement volume 112 and other objects.

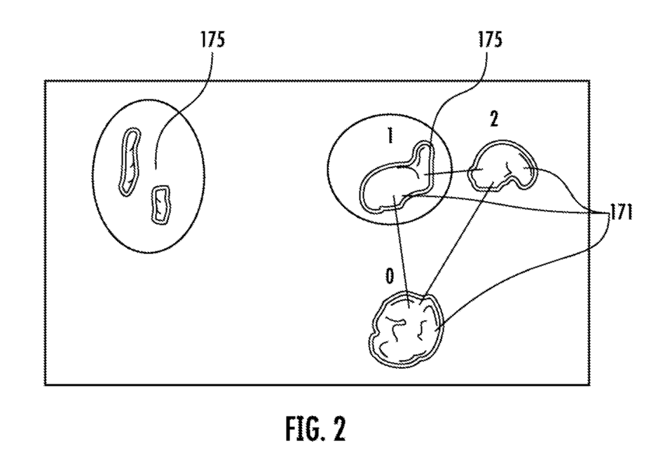

FIG. 2 shows a digitized rendering of an optical image of three markers 171 following automated boundary recognition segmentation analysis. The digitized rendering includes specular reflection 175. Using “selective detection” the filters on the cameras would eliminate this remnant from optical image itself.

In one embodiment of the invention, a method for motion corrected tomographic imaging includes the steps of illuminating a region of interest (ROI), the ROI being part of an unrestrained live subject 160 and having at least three spaced apart optical markers 171 thereon. Simultaneous images can be acquired from different positions by at least a first 116 and a second camera 116 of the markers 171. Motion data comprising 3D position and orientation (pose) of the markers 171 relative to an initial reference position is then calculated from the simultaneous images. Using the motion data, corrected tomographic data is obtained from the ROI, wherein motion corrected tomographic images are obtained therefrom.

An embodiment of the inventive method is now described. A pair of stereoscopically oriented cameras 116 acquires a synchronized pair of images so that each pair of images consists of two views of an arrangement of the markers 171 taken simultaneously. For each stereo pair of images acquired by the cameras 116, an algorithm is used to locate the markers 171 in each of the two images and calculate their position and orientation in three-dimensional space relative to the cameras 116. If the markers 171 are affixed to a rigid body, then the configuration of the reflectors 171 seen by the cameras 116 can be directly translated to the relative pose of the body. The algorithms used to calculate pose in this method are fast enough that pose measurements can be performed in real time while the subject 160 is undergoing a tomographic scan, allowing for immediate notification to the user if any tracking problems are encountered during the scan. The pose data is recorded to a file with a global timestamp and can later be merged with corresponding time-stamped tomographic scan data to correct for any motion in order to tomographically reconstruct an accurate depiction of the scanned area.

An exemplary system based on the invention been demonstrated for a single photon emission computed tomography (SPECT) scanner for performing awake animal imaging while compensating for motion of the subject during the scan. SPECT is one of several nuclear imaging techniques. Generally, in nuclear imaging, a radioactive isotope is injected into, inhaled by or ingested by a subject, such as a patient or other subject. The isotope, provided as a radioactive-labeled pharmaceutical (radio-pharmaceutical) is chosen based on bio-kinetic properties that cause preferential uptake by different tissues. The gamma photons emitted by the radio-pharmaceutical are detected by radiation detectors 128 outside the body, giving the spatial and uptake distribution of the radio-pharmaceutical within the body while minimizing trauma to the subject.

Although described relative to SPECT, the invention is in no way limited to SPECT. For example, the invention is applicable to other tomography methods, such as computed tomography (CT), or positron emission tomography (PET). The invention is also applicable to non-tomography-based scanned imagining, such as MRI or ultrasound. More generally, any application generally requiring 3D motion tracking of a living subject for positioning and correction can benefit from the invention.

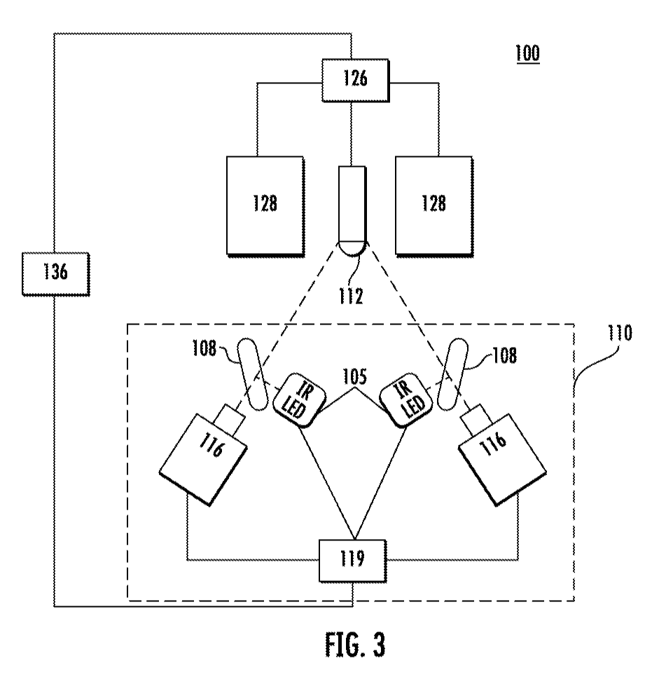

FIG. 3 is a schematic diagram of an exemplary motion correcting SPECT imaging system 100. System 100 includes a motion correcting system 110 that can include IR LED sources 105 for illuminating a restraining volume 112 having a live unrestrained subject, such as a mouse (not shown). As shown in FIG. 1, the mouse can have three spaced apart retro reflective optical markers 171 attached to its head (not shown).

A minimum of three markers is needed to measure both position and orientation of the ROI. Although system 100 is described as having three (3) markers, any number of markers greater two (2) markers may be used if the position and number of optical cameras avoids multiple epipolar solutions. An algorithm for the calculation described below can fit three or more markers to a model. Additional markers will generally improve system robustness. For example, if one or more markers 171 become obscured, as long at least three markers are observed, then a 3D measurement can still be made.

In another approach the system and method can include more than two optical cameras 116. The additional optical cameras 116 can be used to obtain improved resolution or to reduce the number of instances where optical markers 171 become obscured. For example, three optical cameras 171 may be positioned in a triangular orientation. It has been determined that approximately 95% of the time all three markers 171 will be visible to at least two of three optical cameras arranged in a triangular arrangement with the central camera 116 positioned even with, but in front of, the confinement volume 112. This can be improved using additional optical cameras 116. In addition three optical cameras arranged horizontally or vertically in a line orthogonal to the axis of a confinement volume 112 can be used if improved horizontal or vertical resolution is desired. Although not necessary, or even desirable, the optical tracking PC 119 in FIG. 3 is shown as being in front of the confinement volume 112.

Where three markers are used, it is generally preferred that the optical markers 171 not be arranged in an equilateral triangle to eliminate rotational symmetry. Similarly, where more than three optical markers 171 are used, the markers 171 can be arranged to avoid rotational symmetry. Rotational symmetry does not prevent the method from operating, but constrains the rotation. Rotational symmetry can also be avoided by using markers 171 with different polarization characteristics, so that each marker 171 can be identified regardless of rotational configuration of the ROI.

For SPECT imaging, the subject, e.g., a mouse, has a radioactive isotope injected into the region to be imaged. A first camera 116 and second camera 116 are provided for acquiring simultaneous images of the retro-reflective optical markers 171 from different positions. The camera(s) 116 can be high speed digital cameras. Useful high speed digital cameras 116 can have frame rates exceeding about 15 frames/sec to capture live motion. An optical tracking PC 119 includes memory and a processor for calculating motion data including 3D position and orientation of the markers 171 relative to an initial reference position. The initial reference position is arbitrary and can be selected as desired.

The illumination provide by LEDs 105 is shown as being coaxial (on-axis) with the optical axis of cameras 116. Half silvered mirrors 108 provide reflection of IR emitted by LEDs 105 onto the optical axis of cameras 116 and transmission of light from the mouse in confinement volume 112 along the optical axis of cameras 116. This arrangement significantly increases marker 171 intensity in the acquired images. The illumination is preferably strobed and the cameras 116 are simultaneously triggered to stop motion during exposure when acquiring simultaneous images from each camera 116.

Although this discussion describes the system 100 primarily using IR LEDs as the illumination source, other sources are possible. In general, IR radiation in the range from 400-1000 nanometers is used. Infrared illumination can be useful because it is not perceived by the subject even if it is strobed. This allows imaging of the subject in a more natural state.

System 100 includes a motion control PC 126 which includes memory. Where necessary, motion control PC 126 can control the relative motion of the mouse burrow 112 and SPECT detectors 128 in conjunction with a suitable gantry structure for rotating mouse burrow 112 (not shown). In some embodiments, sufficient detectors 128 may be present that rotation is not necessary, while in others, a plurality of detectors will be used in order to reduce the necessary angle of rotation and expedite acquisition of the SPECT data.

The radiation detectors 128 can also include a specially designed collimator to acquire data from different projection views. The system 100 can also include a SPECT data acquisition PC 136 having memory. The data acquisition PC 136 can receive the motion data comprising 3D position and orientation of the markers 171 relative to an initial reference position from PC 119, and correct the radiation data received from the radiaiton detectors 128 for motion of the subject 160. Although described as having three PCs, the invention can use one or more other processor and memory comprising devices for functions provided by PCs 119, 126 and 136. In addition, the functions of the PCs can be combined and provided by fewer than three processors, PCs or combination of both. Although wired communications links are shown in FIG. 1, the invention is in no way limited to this arrangement. For example, communications can be optical or over the air, e.g., radio frequency (RF).

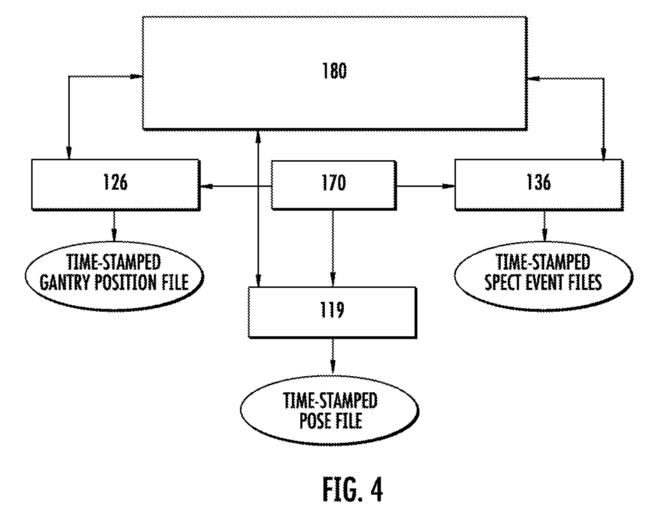

FIG. 4 shows a communication flow diagram based on the system 100 of FIG. 3. A system clock 170 (common for the whole system) provides timing information to motion control PC 126, optical tracking PC 119, and SPECT data acquisition PC 136. The respective PCs time stamp image data obtained for storage therein. SPECT gantry 180 exchanges position information with PC 119.

Returning to the sample method, the first step in motion correction according to the invention is to measure the motion of the ROI to be imaged. The subject 160 can be confined but otherwise unrestrained, such as in a cylindrical burrow 112 with a hemispherical front. The burrow 112 is transparent to the illumination wavelength, which can be a LED emitting electromagnetic energy at 830 nm. This near IR wavelength is invisible to the subject 160 and thus should cause no distraction to the subject being imaged. In addition, the burrow 112 is optically uniform so that external images of the animal can be made without significant distortion. Accurate measurement of position and orientation of the subject is required. The system must also process images fast enough to follow any motion smoothly without gaps especially when fast, jerky movements are encountered.

Although described for use with an animal, the claimed method and system can be used in connection with imaging humans, particularly children or individuals who are unable to remain still for the duration of the tomography scan. Images of humans will generally be acquired without the need for a confinement volume.

The inventive system and related method has been demonstrated to accurately measure head motion of mice using optical markers 171 placed on the head. The cameras 116 shown in FIG. 3 were configured with optical CMOS cameras with 512 by 512 pixel resolution viewing the head from different positions to image the markers 171 and then calculate the 3-D position and orientation of the markers 171 with respect to an initial (reference) position.

The cameras 116 are preferably initially calibrated both intrinsically and extrinsically. Intrinsic calibration involves calculating the lens focal length, optical center, and lens distortion. Extrinsic calibration involves calculating the position and orientation of a calibration pattern with respect to the camera frame of reference. A stereo calibration is then performed to calculate the position and orientation of one camera with respect to the other(s). With this calibration and the measurement technique, measurement accuracies within 100 micrometers in position and 0.1 degrees in rotation can be obtained.

For the prototype SPECT system tested, system speed was limited by the CMOS cameras 116 and hardware in PC 119 to about 15 measurements/sec. Faster speeds can be obtained through higher frame rate cameras and higher performance PC hardware. However, this rate has been found sufficient to smoothly track mouse motion.

The method and device can also include tracking of live video of the camera(s) 116 without requiring strobed illumination. Using constant illumination, a fixed exposure time is set in the camera(s) that is sufficiently short to ensure that motion blurring will not be present in the images. All video images are acquired using this exposure setting.

In a preferred embodiment, the below listed steps are preferably performed sequentially in computing the position and orientation of the ROI, e.g., the head of the animal.

1. Each camera 116 acquires simultaneous images of the head of the subject 160. The illumination is strobed to millisecond or sub-millisecond duration to freeze the motion of the ROI. The image acquisition from the camera(s) 116 is synchronized to the strobe pulse to cause the simultaneous acquisition of optical images during the illumination period.

2. Each image is processed to extract the marker 171 positions by a combination of segmentation, object features extraction and digital filtering. An image processing reference for basic image segmentation (including region growing), feature extraction, and digital filtering is Digital Image Processing, Gonzalez and Woods, 2nd Edition, Prentice Hall, 2002. This step can use a region growing algorithm to segment the markers 171 along with connected component analysis to extract shape and size parameters. Segmentation uses a region growing image thresholding method to separate the markers 171 from the background. Connected component analysis identifies the separate markers 171, labels them, and calculates the location, size, aspect ratio, and other parameters for each marker 171. Due to reflections from the burrow 112, false segmentations can occur where polarization techniques are not employed. The false segmentations can be removed through a combination of shape and size filtering as well as model fitting described below. In the alternative, these reflections can be removed using the polarization techniques described herein. Digital filtering is performed on these geometric parameters to ensure that only true markers are identified. Digital filtering can be enhanced by using markers with a shape distinguishable from specular reflections, e.g., a hemispherical shape. For the special case where a reflection merges with a true marker, the contour can be analyzed for roundness and convexity to identify the true marker.

3. Marker correspondence is performed using the fundamental matrix and epipolar line geometry. As a suitable reference for this step, see R. Hartley, A. Zisserman, Multiple View Geometry in Computer Vision, Cambridge: Cambridge University Press, 2000. Where three markers are used, the fundamental matrix is a 3 by 3 matrix that is an algebraic representation of epipolar geometry. Epipolar geometry is the intrinsic projective geometry used to resolve the position of objects in three-dimensional space. A property of epipolar line geometry is that corresponding points in stereo images line on the same epipolar line. The Hartley reference defines these terms. Use is made of this property in finding corresponding points and in accurately positioning the centroids of corresponding points to the nearest epipolar line. A 3-D point that is imaged by both cameras 116 lies on corresponding epipolar lines.

4. Marker locations are corrected to lie on the nearest epipolar line to improve accuracy. The closest point on the epipolar line is computed from each image location.

5. The 3-D locations for each of the markers are now calculated. Based on the known geometry, the point of closest distance of the two 3-D lines from each camera image point and the optical center are computed. These are generally skew lines that do not intersect.

6. The markers are then fitted to a model. Geometric relationships and fitting error are used to choose the best fit of the markers to the model. This fitting can occur even with additional false markers present. The fitting can use three or more marker positions. The 3D coordinates of at least three markers are known in two reference frames: the camera reference frame and the model reference frame. What is unknown is the correspondence of the points between the reference frames. All valid geometric permutations of correspondence of points between the two frames are calculated using Horn's method (See B. Horn, “Closed-form solution of absolute orientation using unit quaternions,” Journal of the Optical Society of America, 4, pp 629-642, April 1987). Horn's method calculates a 3D transformation and fitting error. The transformation that has the minimum fitting error and that satisfies the geometric constraints is selected. The geometric constraints require that the physical arrangement of points be the same in the two reference frames and that the model is facing the camera as an example.

7. The relative orientation of points between camera reference and model reference are then calculated. Horn's method (referenced above) can be used to calculate the relative orientation between the two reference frames.

8. The position and orientation of the ROI relative to an initial reference position is then calculated.

A preferred embodiment where one camera 116 and at least four optical markers 171 are used will be similar to the eight step process outlined above, except that steps 3 and 4 are replaced by a step where the 2D homography of the four optical markers 171 in each processed image is calculated. The position and orientation of the four optical markers 171 is then calculated using the 2D nomography. Although not required, the process can include (i) attaching the optical markers 171 in a planar orientation, (ii) assuming that the four optical markers 171 form a plane, or (iii) both. As a suitable reference for this step, see R. Hartley, A. Zisserman, Multiple View Geometry in Computer Vision, Cambridge: Cambridge University Press, 2000.

The invention thus includes a motion tracking system and related method which provide robust, reliable measurement of 3D position and orientation of a ROI where motion is present and where point features can be located in optical images. The invention is able to rapidly monitor and correct image data for motion, including motion that is neither smooth nor continuous. Accurate position measurements better than 0.1 mm have been made within the volume of a 75 mm cube. Moreover, objects within a transparent enclosure can be measured even where the view of the object is limited due to obstructions.

The invention can benefit any application generally requiring 3D motion tracking of a living subject for positioning and motion correction. Motion tracking systems according to the invention can thus be used in a wide variety of tomographic imaging systems which require exacting alignment, particularly when the region of interest is moving (or can move) during the measurement or other procedure. Significantly, using optical methods according to the invention to track the position of the subject during a scan, the physiology of the subject can be kept free from physical and chemical effects that are otherwise necessary for high quality imaging. Such techniques can interfere with the control of conventional pharmaceutical screening processes.

As noted above, the invention can be applied to SPECT, other tomography, such as computed tomography (CT) and positron emission tomography (PET), as well as non-tomography-based scanned imagining, such as MRI or ultrasound. The invention can be integrated into new systems as well as be used to retrofit existing systems.

### EXAMPLES

It should be understood that the Examples described below are provided for illustrative purposes only and do not in any way define the scope of the invention.

### Example #1

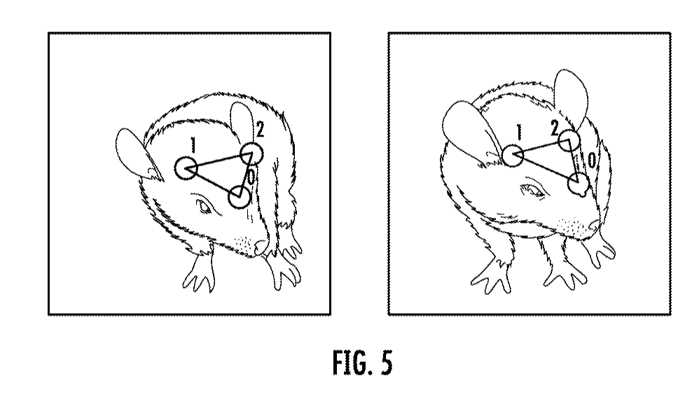

FIG. 1 shows an optical image of a mouse 160 fitted with three optical retro-reflective markers 171 on its head in a burrow 112. Images of the mouse with the retro reflectors from each camera and with tracking enabled are shown in FIG. 5. Tracking is shown by lines connecting the center of each marker 171. Also visible are reflections off the glass tube enclosure that have been ignored. The markers are outlined and numbered showing that they have been segmented and that correspondence has been determined. In this depiction, the lines between the markers 171 indicate that successful model fitting has been performed and that a full 3D transformation has been calculated between the camera reference frame and the model reference frame.

### Example #2

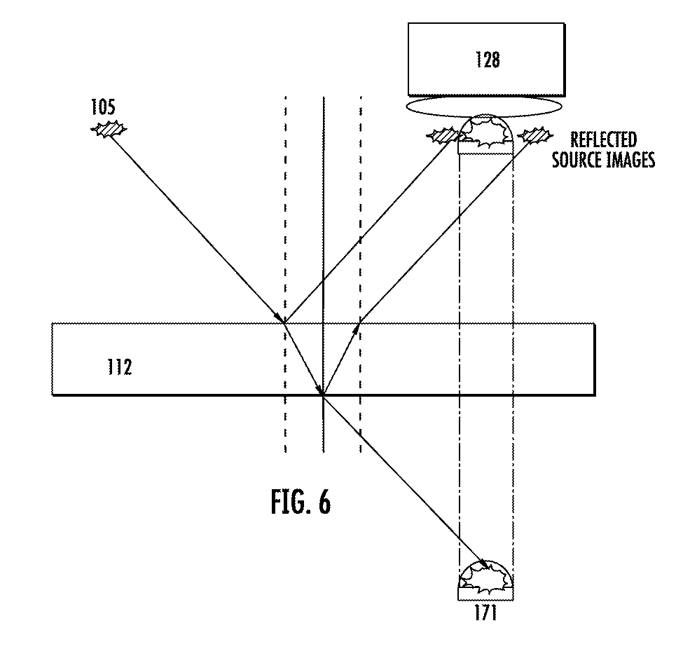

As described above, specular reflections 175 can make it difficult to discern markers 171. This problem is apparent from the specular reflections 175 in FIG. 2 and the illustration in FIG. 6. One way to eliminate specular reflections is the use of polarized light, polarizing filters, or both. Maulus' law for determining polarized light intensity is:

I(θ)=Iₒ cos²(θ) (1)

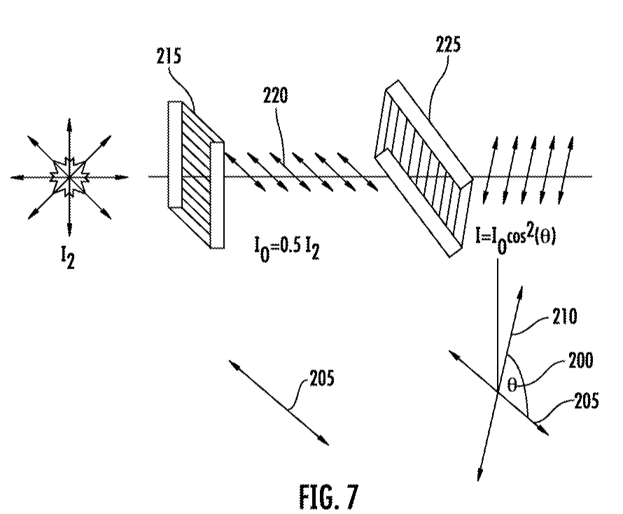

In Equation 1, I(θ) is the detected intensity, which is related to the input light intensity, Iₒ, by a factor of the square of the cosine of the relative angle (θ) 200 between the input light state-of-polarization (SOP) axis 205 and the transmission axis 210 of the preceding polarizer (polarization filter), as shown in FIG. 7.

In FIG. 7, an unpolarized light beam of radiation, Iₛ (e.g., infrared radiation) is passed through a linear polarizer 215 where it looses half of its original intensity. This can be explained by the assumption an unpolarized light source can be decomposed into two orthogonal polarization vectors, which upon summation give a net polarization vector of zero (i.e., no preferred polarization state). In the terms of Equation 1, half of the initial unpolarized light would be blocked because θ=90° for one of any two orthogonal polarization vectors used to characterize unpolarized light. Having passed through the linear polarizer 215, the now polarized light beam (Iₒ) 220 propagates through the system and encounters additional polarizer(s) 225. The intensity, as calculated using Equation 1 and the SOP is always coincident with the transmission axis of the preceding polarizer.

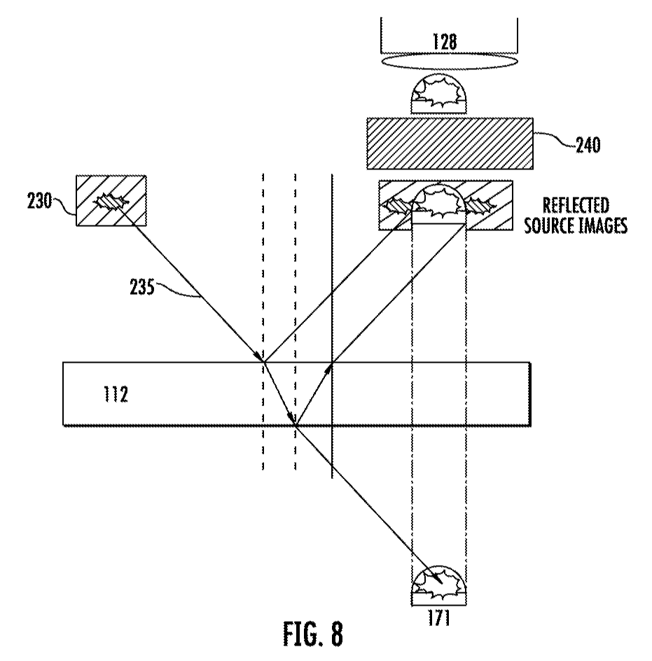

As shown in FIG. 8, Maulus' law can be used to develop a polarization filter for selective detection of the markers 171. For example, an input light source can be passed through a first polarization filter 230 to provide polarized illumination 235. A second polarization filter 240 that is aligned crossed polarized, i.e., θ=90°, to the polarized illumination 235 can be placed in front of an imaging camera 116. Such an arrangement can produce images completely free of specular reflections 175 from surfaces other than optical markers 171, such as a test tube 112.

For purposes of optical marker tracking using a polarization filter approach, the retro-reflective optical markers can either (i) depolarize the incident SOP, which allows 50% of the retro-reflected light through the polarization filter, (ii) rotate the plane of linear polarization between 0° and 90° in order to allow for some of the retro-reflected light to make it through the polarization filter, or (iii) both. The optical markers can rotate the plane of linear polarization between 5° and 85°, or between 10° and 80°, or between 15° and 75°, or between 30° and 60°, or any combination thereof, such as between 10° and 60°.

One method of preventing rotational symmetry is to use at least two retro-reflective markers 171 that have different polarization characteristics. Polarization characteristics include, but are not limited to, polarizating, depolarizating, unpolarized, and polarization rotation. For example, two markers 171 having different angles of polarization rotation have different polarization characteristics.

This invention can be embodied in other forms without departing from the spirit or essential attributes thereof and, accordingly, reference should be had to the following claims rather than the foregoing specification as indicating the scope of the invention.

We claim:

1. A method for generating motion corrected tomographic images, comprising the steps of:
illuminating a region of interest (ROI) to be tomographically imaged, wherein said ROI is part of an unrestrained live subject having at least three spaced apart retro-reflective optical markers attached thereto, wherein said markers are proximate said ROI and each marker is either polarizing or depolarizing for an illuminating wavelength;
acquiring filtered optical images of said markers from at least one filtered camera, wherein a polarization filter on the at least one filtered camera enables selective detection of illumination reflected by the at least three optical markers;
calculating motion data comprising 3D position and orientation of said markers relative to an initial reference position, wherein the at least three spaced apart retro-reflective optical markers and the at least one filtered camera are sufficient in quantity and position to avoid multiple epipolar solutions; and
motion correcting tomographic data of said ROI obtained simultaneously with said motion data using said motion data, wherein motion corrected tomographic images are obtained.

2. The method of claim 1, wherein during said method said animal is disposed in a confinement volume which is optically transparent to said illumination wavelength.

3. The method of claim 1, wherein said tomographic images are single photon emission computed tomography (SPECT) images.

4. The method of claim 1, wherein said illumination is aligned to be approximately coaxial with an optical axis of said first camera.

5. The method of claim 1, wherein said illuminating is strobed illuminating.

6. The method of claim 5, wherein said calculating motion data step comprises processing said images using a combination of segmentation, object feature extraction and digital filtering.

7. The method of claim 1, further comprising at least a fourth spaced apart retro-reflective optical marker, wherein at least four of the markers are arranged to eliminate multiple epipolar solutions.

8. The method of claim 1, comprising a first and a second filtered camera, wherein the acquiring step comprises acquiring simultaneous images from the first and second filtered cameras.

9. The method of claim 8, wherein said calculating motion data step comprises processing said simultaneous images using a combination of segmentation, object feature extraction and digital filtering.

10. The method of claim 1, comprising a first, a second, and a third filtered camera, wherein the acquiring step comprises acquiring simultaneous images of at least three of the spaced apart retro-reflective optical markers from at least two of the first, second, and third filtered cameras.

11. The method of claim 1, wherein said illumination is polarized.

12. The method of claim 1, wherein at least two of said markers are polarized and have different polarization characteristics.

13. The method of claim 1, wherein at least one of the at least one filtered cameras is a video camera.

14. A motion correcting tomography-based imaging system, comprising:
at least three spaced apart retro-reflective optical markers for placement on a region of interest (ROI) to be imaged, wherein each of said markers is either polarizing or depolarizing for a wavelength produced by an illumination source;
at least one radiation detector for collecting radiation data emitted from a radioactive isotope in said ROI or radiation data provided by said ROI attenuating radiation provided by an external radiation source, and a first processor communicably connected to said radiation detector, and
structure for positioning said radiation detector relative to said ROI, and
a motion correcting system, comprising:
at least one illumination source for illuminating said ROI;
at least one filtered camera for acquiring images from said markers,
structure for positioning said at least one filtered camera; and
at least a second processor communicably connected to said first processor for calculating motion data comprising 3D position and orientation of said markers relative to an initial reference position, and motion correcting said radiation data, wherein motion corrected tomographic images are obtained from said motion correcting radiation data, wherein said system comprises at least two filtered cameras, at least four spaced apart retro-reflective optical markers, or both, in order to avoid multiple epipolar solutions.

15. The system of claim 14, wherein said system is a single photon emission computed tomography (SPECT) system.

16. The system of claim 15, wherein said illuminating is aligned to be approximately coaxial with at least one of said at least one filtered cameras.

17. The system of claim 16, wherein said at least one illumination source provides strobed illumination.

18. The system of claim 17, wherein acquisition of said images is synchronized to a strobe pulse to cause the simultaneous acquisition during an illumination period.

19. The system of claim 14, wherein said at least one radiation detector comprises a first and a second detector.

20. A method for generating motion corrected tomographic images, comprising the steps of:
illuminating a region to of interest (ROI) to be imaged being part of an unrestrained live subject and having at least three spaced apart optical markers thereon;
acquiring optical images of said markers from at least one camera;
calculating motion data comprising 3D position and orientation of said markers relative to an initial reference position, wherein the at least three spaced apart optical markers and the at least one camera are sufficient in quantity and position to avoid multiple epipolar solutions, and
motion correcting tomographic data obtained from said ROI using said motion data, wherein motion corrected tomographic images are obtained.

21. A motion correcting tomography-based imaging system, comprising:
at least three spaced apart optical markers for placement on a region of interest (ROI) to be imaged;
at least one radiation detector for collecting radiation data from emitted from a radioactive isotope in said ROI or radiation data provided by said ROI attenuating radiation provided by an external radiation source, and a first processor communicably connected to said radiation detector, and
structure for positioning said radiation detector relative to said ROI, and
a motion correcting system, comprising:
an at least one illumination source for illuminating said ROI;
at least one camera for acquiring images of said markers, and
at least a second processor communicably connected to said first processor for calculating motion data comprising 3D position and orientation of said markers relative to an initial reference position, and motion correcting said radiation data, wherein motion corrected tomographic images are obtained from said motion correcting radiation data, wherein the system includes at least two filtered cameras, at least four spaced apart retro-reflective optical markers, or both, in order to avoid multiple epipolar solutions.
